(19) United States
(12) Patent Application Publication
(10) Pub. No.: US 2023/0233176 A1
(43) Pub. Date: Jul. 27, 2023

(54) SYSTEM AND METHOD OF CALIBRATING CEREBRAL SENSOR ORIENTATION AND GENERATING FEEDBACK FROM CEREBRAL SENSOR INJECTOR

(71) Applicant: NovaSignal Corp., Los Angeles, CA (US)

(72) Inventors: Robert Hamilton, Los Angeles, CA (US); Corey Thibeault, Los Angeles, CA (US); Shankar Radhakrishnan, Los Angeles, CA (US); Michael O'Brien, Los Angeles, CA (US); Mina Ranjbaran, Los Angeles, CA (US); Gerard Salinas, Los Angeles, CA (US); Naresh Sehgal, Los Angeles, CA (US); Jayarajasekaran Janarthanan, Los Angeles, CA (US)

(21) Appl. No.: 17/581,809

(22) Filed: Jan. 21, 2022

(51) Int. Cl. A61B 8/08; A61B 8/00

(52) U.S. Cl. CPC A61B 8/0808; A61B 8/488; A61B 8/58; A61B 8/4254; A61M 5/178

(57) ABSTRACT

Example implementations can include a method of calibrating orientation of a cerebral sensor and generating feedback from an injector for a cerebral sensor, by scanning an anatomical structure, by a cerebral sensor, proximate to the scan position and satisfying a first threshold, obtaining at least one cranial signal from the cerebral sensor in accordance with a first pathway over the anatomical structure, obtaining from the cranial signal a point corresponding to the anatomical structure, storing the obtained point to a point cache operable to store one or more points, in response to a determination that the point satisfies a second threshold, modifying the first pathway for actuating the cerebral sensor, in response to the determination that the obtained point satisfies the second threshold, selecting an optimized point from the point stack, and actuating the cerebral sensor continuously based on the optimized point.

### TECHNICAL FIELD

The present disclosure relates generally to ultrasound devices, and more particularly to calibrating orientation of a cerebral sensor and generating feedback from an injector for a cerebral sensor.

### BACKGROUND

Clinical guidelines recommend monitoring medical conditions including stroke, emboli, stenosis, vasospasm as well as elevated intracranial pressure (ICP) which may alter cerebral blood flow. Right to Left shunt (RLS) through a Patent Foramen Ovale (PFO) is a known risk factor of ischemic stroke and is present in approximately 25% of the entire population. The American College of Cardiology recognizes the benefit of procedures to close PFO in patients with PFO-associated stroke for secondary stroke prevention. With the connection between PFO closure and prevention of secondary stroke, there is a need for assessment tools that are both non-invasive and accurate.

### SUMMARY

Present implementations are directed to calibration of an orientation of a cerebral sensor, with respect to a patient's cranial features and with respect to an injection process associated with sensing by the cerebral sensor. Present implementations can advantageously align a cerebral sensor with an optimized surface position on a portion of a patient's body, and depth position at a particular depth below the surface position, based on feedback from multiple sources including patient treatment activity to prepare the patient for a medical diagnostic including the cerebral sensor.

Present implementations can automate setup, acquisition, protocol conduct, interpretation, and automated treatment monitoring and feedback at least for patients with Right-to-Left (RLS) vascular shunt. Automated treatment monitoring and feedback can advantageously result in the technical solution of alignment of sensor probes to an anatomical structure of a patient's body, to solve the technical problem of lack of effective transcranial Doppler (“TCD”) sensor feedback in RLS detection applications. Advantages of present implementations can include at least rapidly collecting diagnostically significant cerebral blood flow velocity (CBFV) for the detection and identification of an RLS shunt (cardiac, pulmonary, or other) using Pulsed Wave (PW) Doppler Ultrasound, accurate and automated conduct of a sensor calibration protocol with effective timing and protocol accuracy feedback to an administering user, including a medical provider. Advantages also can include rapid and highly accurate analysis of the results of data collected from a procedure providing detection and identification of the RLS shunt or type of RLS shunt in the patient. Thus, a technological solution for calibrating orientation of a cerebral sensor and generating feedback from an injector for a cerebral sensor is provided.

A method can include calibrating orientation of a cerebral sensor and generating feedback from an injector for a cerebral sensor, by scanning an anatomical structure, by a cerebral sensor, proximate to the scan position and satisfying a first threshold, obtaining at least one cranial signal from the cerebral sensor in accordance with a first pathway over the anatomical structure, obtaining from the cranial signal a point corresponding to the anatomical structure, storing the obtained point to a point cache operable to store one or more points, in response to a determination that the point satisfies a second threshold, modifying the first pathway for actuating the cerebral sensor, in response to the determination that the obtained point satisfies the second threshold, selecting an optimized point from the point stack, and actuating the cerebral sensor continuously based on the optimized point.

In some arrangements, the method further includes generating a second pathway over the anatomical structure iteratively at a second granularity different from a first granularity associated with the first pathway.

In some arrangements, a first threshold corresponds to one or more metrics associated with one or more of a surface position and a depth of the scan position with respect to the anatomical structure.

In some arrangements, a first threshold corresponds to one or more metrics associated with the anatomical structure.

In some arrangements, the cranial signal includes a transcranial Doppler signal.

In some arrangements, a second threshold corresponds to at least one of an M-mode component of the transcranial Doppler signal, a spectrogram component of the transcranial Doppler signal, an envelope component of the transcranial Doppler signal, and an IQ-pair component of the transcranial Doppler Signal.

In some arrangements, the method further includes receiving feedback from an injection kit, in response to an actuation of an injector associated with the anatomical structure, and presenting, at a user interface, a presentation based on the feedback from the injection kit.

In some arrangements, feedback with a pressure value corresponding to a magnitude of pressure is applied to the injector.

In some arrangements, a presentation comprises an indication corresponding to a pressure applied to the injector.

In some arrangements, a system of calibrating orientation of a cerebral sensor and generating feedback from an injector for a cerebral sensor, can include a sensor controller to scan an anatomical structure, by a cerebral sensor, proximate to the scan position and satisfying a first threshold, obtain at least one cranial signal from the cerebral sensor in accordance with a first pathway over the anatomical structure, and actuate the cerebral sensor continuously based on an optimized point, a point feedback processor operatively coupled with the sensor controller, the point feedback processor to obtain from the cranial signal a point corresponding to the anatomical structure, and to store the obtained point to a point cache operable to store one or more points, in response to a determination that the point satisfies a second threshold, and a scan controller to modify the first pathway for actuating the cerebral sensor, in response to the determination that the obtained point satisfies the second threshold, and select the optimized point from the point stack.

In some arrangements, the system can include a scan controller to generate a second pathway over the anatomical structure iteratively at a second granularity different from a first granularity associated with the first pathway.

In some arrangements, the first threshold corresponds to one or more metrics associated with one or more of a position and a depth of the scan position.

In some arrangements, the first threshold that corresponds to one or more metrics associated with the anatomical structure.

In some arrangements, the cranial signal includes a transcranial Doppler signal.

In some arrangements, the second threshold that corresponds to at least one of an M-mode component of the transcranial Doppler signal, a spectrogram component of the transcranial Doppler signal, an envelope component of the transcranial Doppler signal, and an IQ-pair component of the transcranial Doppler signal.

In some arrangements, the system can include a user interface controller to present, at a user interface, a presentation based on feedback from an injection kit, and s sensor controller to receive the feedback from the injection kit, in response to an actuation of an injector associated with the anatomical structure.

In some arrangements, the feedback that includes a pressure value corresponding to a magnitude of pressure applied to the injector.

In some arrangements, a presentation comprises an indication corresponding to a pressure applied to the injector.

In some arrangements, a computer readable medium can include one or more instructions stored thereon and executable by a processor to cause, by the processor, a cerebral sensor to scan an anatomical structure proximate to the scan position and satisfying a first threshold, obtain, by the processor, at least one cranial signal from the cerebral sensor in accordance with a first pathway over the anatomical structure, obtain, by the processor, from the cranial signal a point corresponding to the anatomical structure, store, by the processor, the obtained point to a point cache operable to store one or more points, in response to a determination that the point satisfies a second threshold, modify, by the processor, the first pathway for actuating the cerebral sensor, in response to the determination that the obtained point satisfies the second threshold, select, by the processor, an optimized point from the point stack, and actuate, by the processor, the cerebral sensor continuously based on the optimized point.

In some arrangements, the first threshold corresponds to one or more metrics associated with one or more of a surface position of the scan position with respect to the anatomical structure, a depth of the scan position with respect to the anatomical structure, a cranial signal that includes a transcranial Doppler signal, and a second threshold that corresponds to at least one of an M-mode component of the transcranial Doppler signal, a spectrogram component of the transcranial Doppler signal, an envelope component of the transcranial Doppler signal, and an IQ-pair component of the transcranial Doppler signal.

### BRIEF DESCRIPTION OF THE DRAWINGS

These and other aspects and features of the present implementations will become apparent to those ordinarily skilled in the art upon review of the following description of specific implementations in conjunction with the accompanying figures, wherein:

FIG. 1 illustrates a system, in accordance with present implementations.

FIG. 2 illustrates a system architecture, in accordance with present implementations.

FIG. 3 illustrates an injector system further to the system architecture of FIG. 2, in accordance with present implementations.

FIG. 4 illustrates a cerebral system further to the system architecture of FIG. 2, in accordance with present implementations.

FIG. 5 illustrates a memory architecture, in accordance with present implementations.

FIG. 6 illustrates a method of optimizing orientation of a cerebral probe, in accordance with present implementations.

FIG. 7 illustrates a method of optimizing orientation of a cerebral probe, in accordance with present implementations.

FIG. 8 illustrates a method of optimizing orientation of a cerebral probe, in accordance with present implementations.

FIG. 9 illustrates a method of optimizing orientation of a cerebral probe, in accordance with present implementations.

FIG. 10 illustrates a method of monitoring and generating feedback of an injection system, in accordance with present implementations.

### DETAILED DESCRIPTION

The present implementations will now be described in detail with reference to the drawings, which are provided as illustrative examples of the implementations so as to enable those skilled in the art to practice the implementations and alternatives apparent to those skilled in the art. Notably, the figures and examples below are not meant to limit the scope of the present implementations to a single implementation, but other implementations are possible by way of interchange of some or all of the described or illustrated elements. Moreover, where certain elements of the present implementations can be partially or fully implemented using known components, only those portions of such known components that are necessary for an understanding of the present implementations will be described, and detailed descriptions of other portions of such known components will be omitted so as not to obscure the present implementations. Implementations described as being implemented in software should not be limited thereto, but can include implementations implemented in hardware, or combinations of software and hardware, and vice-versa, as will be apparent to those skilled in the art, unless otherwise specified herein. In the present specification, an implementation showing a singular component should not be considered limiting. Rather, the present disclosure is intended to encompass other implementations including a plurality of the same component, and vice-versa, unless explicitly stated otherwise herein. Moreover, applicants do not intend for any term in the specification or claims to be ascribed an uncommon or special meaning unless explicitly set forth as such. Further, the present implementations encompass present and future known equivalents to the known components referred to herein by way of illustration.

Transcranial Doppler (TCD) Ultrasound is a non-invasive sonographic method used to evaluate blood flow in a wide variety of conditions. Clinical guidelines recommend monitoring medical conditions including stroke (ischemic and hemorrhagic), traumatic brain injury, headache (including migraine), dementia, sickle cell anemia, brain death, and during surgical procedures which may alter cerebral blood flow. In the case of embolic ischemic stroke or TIA in the absence of clear risk factors, TCD may also be used to aid in the diagnosis of a potential cardiac source of emboli, including conditions such as atrial fibrillation, Right-to-Left shunt, and patent foramen ovale (PFO). Identification of PFO, and subsequent evaluation of severity, can determine the optimal course of management or treatment for prevention of subsequent stroke through PFO-occluding devices. Because closure of large shunts improves secondary stroke prevention, the need for an accurate diagnostic device or system is advantageous. It is to be understood that present implementations are not limited to the example discussed herein.

Transcranial Doppler (TCD) devices can perform non-invasive, cerebral blood flow velocity monitoring using ultrasound which can be used for a number of medical conditions including those listed above. However, displays and screens on conventional TCDs show simple waveforms without any diagnostic visualization that can assist a physician with equipment calibration or diagnosis in real-time.

Acquiring the cerebral blood flow velocity (CBFV) signals using TCD requires placement of a transducer within a specific region of the skull thin enough for the ultrasound waves to penetrate, locating a signal of the artery of interest, and maintaining a steady position for sufficient sample size. The location of these narrow windows varies significantly from person to person. Additionally, reading and interpreting the scans once complete is difficult because subtle features and changes in the CBFV waveforms that indicate neurological disorders are not easily discernible using traditional TCD analysis or visual inspection. These requirements make insonating (i.e., exposing to ultrasound) the desired blood vessel difficult, thus restricting TCD use to major hospitals with expensive, on staff expert human sonographers to operate the device as well as reducing the overall utility of the device through utilization of only simple analysis.

Present implementations can include one or more ultrasound transducers to measure cerebral blood flow velocity (CBFV) information using pulsed wave (PW) Doppler Ultrasonography. CBFV factors can include velocity, emboli, spectrum, audio, m-mode, and raw data containing the in-phase and quadrature components (IQ pairs). Present implementations can also measure the region of insonation, using, for example, angle of insonation, in the case of beam steering, or mechanical or optical encoders. These measurements can use at least the spatial position and orientation of a transducer or transducer array of a sensor probe, along with depth information provided by the PW Doppler, to estimate the sample volume being insonated. Present implementations can include a processing unit which can combine the CBFV and sample volume estimate to direct to system to the optimum sample volume to conduct the RLS diagnostic.

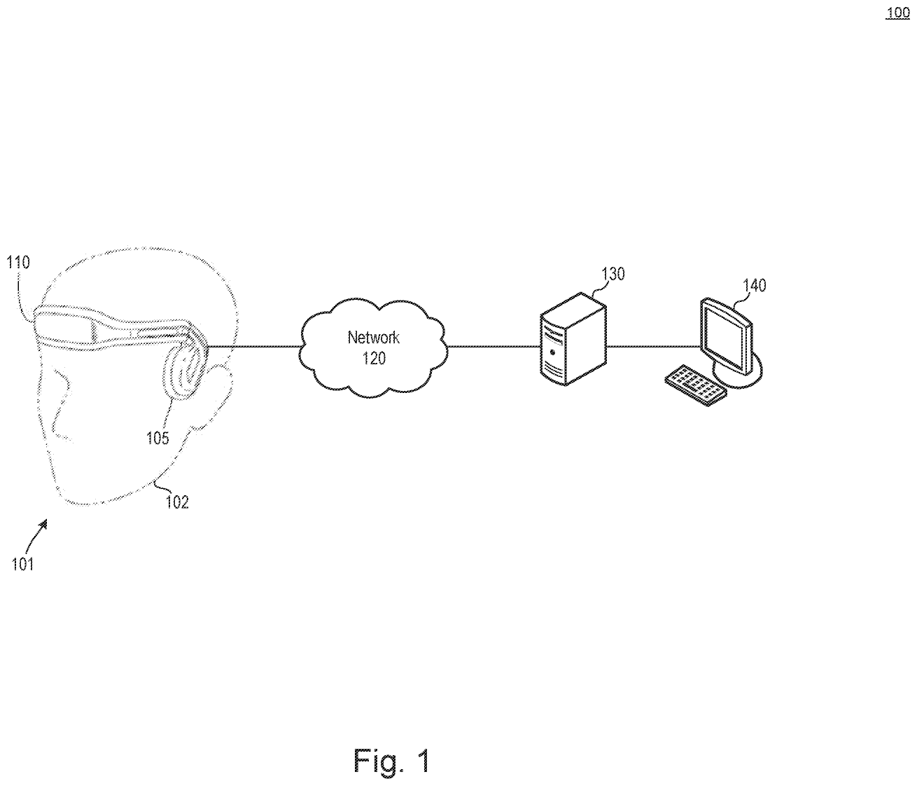

FIG. 1 illustrates a system, in accordance with present implementations. Referring to FIG. 1, the waveform visualization system 100 includes at least a headset device 110, a controller 130, and an output device 140.

The headset device 110 is a TCD ultrasound device configured to emit and measure acoustic energy in a head 102 of a patient 101. The headset device 110 includes at least one probe 105 (e.g., at least one ultrasound probe) configured to emit and measure ultrasound acoustic energy in the head 102. For example, the probe 105 includes at least one TCD scanner, which can automatically locate the middle cerebral artery (MCA) in some arrangements. At least one probe 105 can be positioned in a temporal window region (temple) of the head 102 to collect the ultrasound data. As one example, the probe 105 can be a single probe that is actuated. As another example, the probe 105 can be a series of ultrasound elements that can be steered electronically. In other arrangements, the probe can be positioned over different acoustic windows such as the transorbital window or the suboccipital window. In some arrangements, headset 110 includes two ultrasound probes 105, which can be placed on the temporal window region on both sides of the head 102. A headband, strap, Velcro®, hat, helmet, or another suitable wearable structure of the like connects the two probes in such arrangements. A lubricating gel can be applied between the head 102 and the probe 105 to improve acoustic transmission. Further disclosure regarding examples of the probe 105 that can be used in conjunction with the waveform visualization system 100 described herein can be found in non-provisional patent application Ser. No. 15/399,648, titled ROBOTIC SYSTEMS FOR CONTROL OF AN ULTRASONIC PROBE, and filed on Jan. 5, 2017, which is incorporated herein by reference in its entirety.

The controller 130 is configured to receive the ultrasound data outputted by the headset device 110 and to generate CBFV waveforms that correspond to the ultrasound data. In that regard, the probe 110 is operatively coupled to the controller 130 via a suitable network 120 to send the ultrasound data to the controller 130. The network 120 can be wired or wireless (e.g., 802.11X, ZigBee, Bluetooth®, Wi-Fi, or the like). The controller 130 can further perform signal processing functions to determine and display morphological indicators corresponding to the CBFV waveforms to facilitate a physician, clinician, technician, or care provider with diagnosis and/or to adjust the positioning of the headset device 110 and the probe 105. Further, as described, the headset device 110 can automatically adjust the position and orientation of the probe 105 responsive to determination that the probe 105 is not optimally placed based on the morphological indicators in the manner described herein. The controller 130, the output device 140, and a portion of the network 120 can be incorporated into a single device (e.g., a touchscreen tablet device).

The output device 140 can include any suitable device configured to display information, results, messages, and the like to an operator (e.g., a physician, clinician, technician, or care provider) of the waveform visualization system 100. For example, the output device 140 includes but is not limited to, a monitor, a touchscreen, or any other output device configured to display the CBFV waveforms, the morphology indicators, and the like for facilitating diagnosis and/or the positioning of the headset device 110 and the probe 105 relative to the head 102 in the manner described.

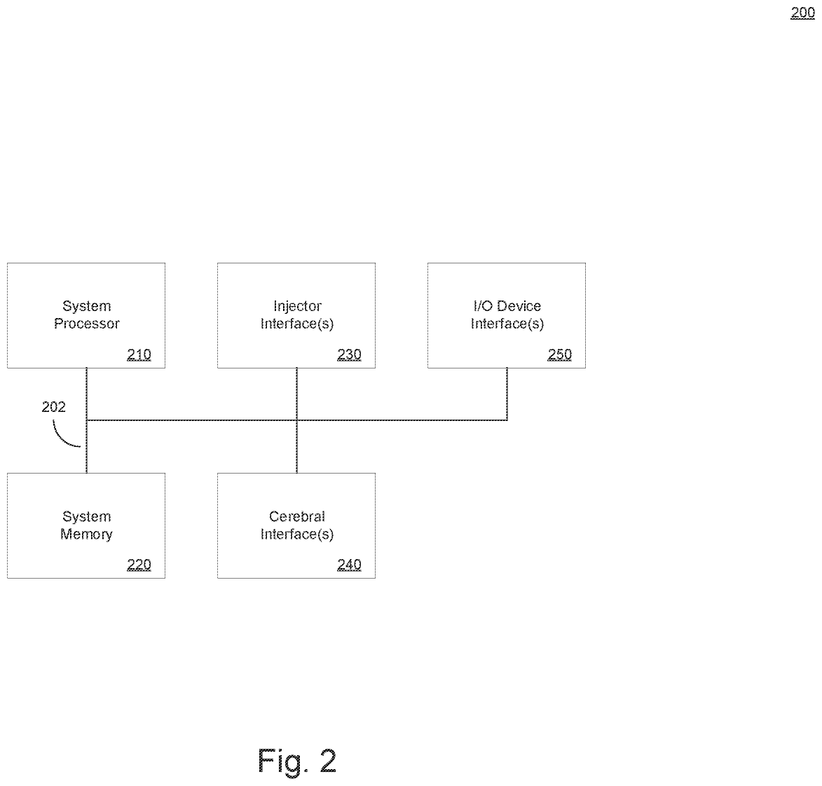

FIG. 2 illustrates a system architecture, in accordance with present implementations. As illustrated by way of example in FIG. 2, system 200 can include a system processor 210, a system memory 220, one or more injector interfaces 230, one or more cerebral interfaces 240, one or more input and output device interfaces 250, and a system communication channel 202. The system 200 can be housed at least partially within the controller 130.

The system processor 210 can execute one or more instructions associated with the system 200. The system processor 210 can include an electronic processor, an integrated circuit, or the like including one or more of digital logic, analog logic, digital sensors, analog sensors, communication buses, volatile memory, nonvolatile memory, and the like. The system processor 210 can include, but is not limited to, at least one microcontroller unit (MCU), microprocessor unit (VIPU), central processing unit (CPU), graphics processing unit (GPU), physics processing unit (PPU), embedded controller (EC), or the like. The system processor 210 can include a memory operable to store or storing one or more instructions for operating components of the system processor 210 and operating components operably coupled to the system processor 210. The one or more instructions can include at least one of firmware, software, hardware, operating systems, embedded operating systems, and the like. The system processor 210 or the system 200 generally can include at least one communication bus controller to effect communication between the system processor 210 and the other elements of the system 200.

The system memory 220 can store data associated with the system 200. The system memory 220 can include one or more hardware memory devices to store binary data, digital data, or the like. The system memory 220 can include one or more electrical components, electronic components, programmable electronic components, reprogrammable electronic components, integrated circuits, semiconductor devices, flip flops, arithmetic units, or the like. The system memory 220 can include at least one of a non-volatile memory device, a solid-state memory device, a flash memory device, and a NAND memory device. The system memory 220 can include one or more addressable memory regions disposed on one or more physical memory arrays. A physical memory array can include a NAND gate array disposed on, for example, at least one of a particular semiconductor device, integrated circuit device, and printed circuit board device.

The injector interfaces 230 can operatively couple the system processor 210 to one or more sensors associated with one or more injector devices or systems. The injector devices or systems can include at least one physical interface to physically couple an injector device to the system 200. The injector interfaces 230 can include at least one physical interface to communicatively couple one or more sensors of an injector device to the system processor 210. The sensors of the injector devices can be associated with an actuator or other moveable component of the injector device. The sensors of the injector devices can be associated with a structure or other fixed component of the injector device. Thus, the injector interfaces 230 can obtain feedback from multiple portions of multiple injector devices by the injector interfaces. The injector interfaces 230 can thus advantageously monitor injector devices and obtain one or more characteristics of the injector devices in real-time, including moveable components of and injector device and fixed structure of the injector device. Real-time can include, for example, during and contemporaneously with a procedure to inject a patient with one or more substances by at least one of the injector devices.

The cerebral interfaces 240 can operatively couple the system processor 210 to one or more sensors and actuators associated with one or more sensor probes or systems. The sensor probes or systems can include at least one physical interface to physically couple a sensor probe to the system 200. A sensor probe can correspond to the probe 105. The cerebral interfaces 240 can include at least one physical interface to communicatively couple one or more sensors and actuators of a sensor probe to the system processor 210. The sensors of the sensor probe can be associated with a TCD sensor, and the TCD signal obtained from the TCD sensor can be responsive to and modifiable based on the presence and magnitude of presence of substance detected within a patient. The substance can correspond to a substance injected into a patient by an injector device associated with the cerebral interfaces 240. The TCD signal and include a CBFV waveform as discussed above. The CBFV waveform can include one or more of an M-mode component, spectrum component, envelope component, and IQ pairs. Thus, the cerebral interfaces 240 can obtain feedback from multiple sensor probes with respect to multiple TCD components, and can provide actuation instructions to articulate or move, for example, one or more of the sensor probes. Articulating and moving a sensor probe can include moving a physical position of the sensor probe with respect to a fixed coordinate system or with respect to an anatomical structure of a patient proximate to the sensor probe. As one example, the anatomical structure can include a cranial landmark, a blood vessel, a skin surface feature, or any portion or feature of the head 102, for example. The cerebral interfaces 240 can thus advantageously obtain sensor feedback from a sensor probe and articulate the sensor probe in real-time to maximize signal acquisition. Real-time can include, for example, during and contemporaneously with a procedure to inject a patient with one or more substances by at least one of the injector devices. The cerebral interfaces 240 can advantageously communicate feedback from the sensor probe responsive to the presence and magnitude of presence of substance detected within a patient, based on feedback received by the TCD signal reacting to, or with respect to, the substance, for example.

The input and output device interfaces 250 can operatively couple the system processor 210 to one or more input device or output devices. The input device can includes, but are not limited to human-computer interface (HCI) devices. HCI devices can include, for example, a computer mouse, a keyboard, capacitive touch interface, resistive touch interface, infrared interface, or the like. The output devices can include, but are not limited to, an electronic display. An electronic display can include, for example, a liquid crystal display (LCD), a light-emitting diode (LED) display, an organic light-emitting diode (OLED) display, or the like.

The system communication channel 202 can communicatively couple the components of the system 200. The system communication channel 202 can communicate one or more instructions, signals, conditions, states, or the like between one or more of the components of the system 200. The system communication channel 202 can include one or more digital, analog, or like communication channels, lines, traces, or the like. As one example, the system communication channel 202 includes at least one serial or parallel communication line among multiple communication lines of a communication interface.

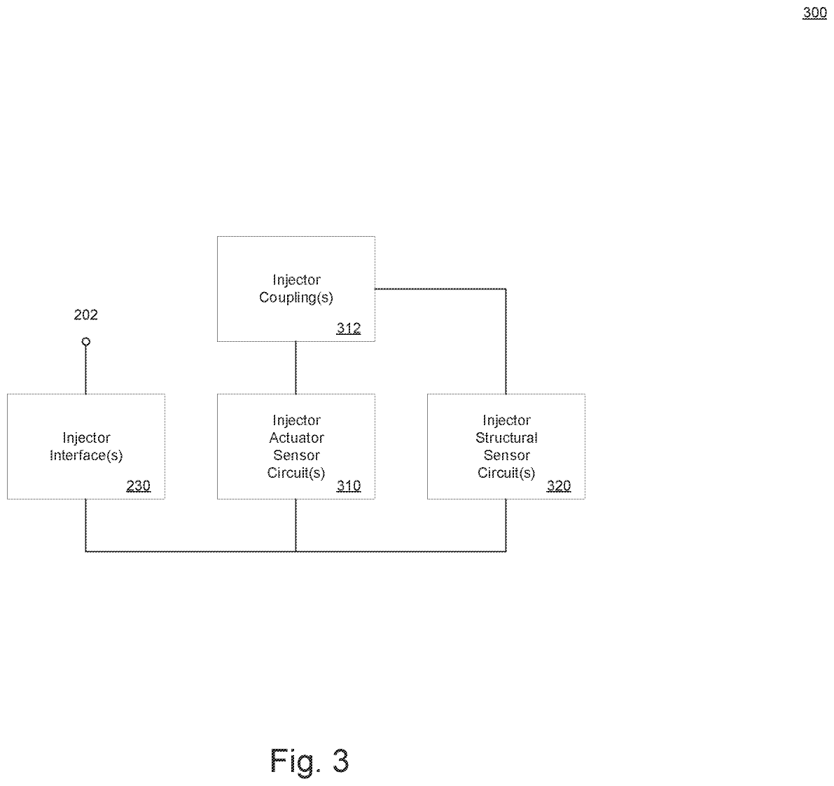

FIG. 3 illustrates an injector system further to the system architecture of FIG. 2. As illustrated by way of example in FIG. 3, an injector system 300 can include the injector interfaces 230, one or more injector actuator sensors 310, one or more injector couplings 312, and one or more injector structural sensor circuits 320.

The injector actuator sensor circuits 310 can detect feedback from at least one sensor operatively coupled with, integrated with, or the like, an injector device. The injector actuator sensor circuits 310 can respond to actuation of at least a moveable portion of an injector. A moveable portion of an injector can include, but is not limited to, a plunger of a syringe. The syringe can be actuated by a medical provider, and the displacement of the plunger can be detected by a sensor among the injector actuator sensor circuits 310 corresponding to the particular injector being operated by the medical provider. It is to be understood that the injector actuator sensor circuits 310 can be located at least partially or entirely within the system 300, or can be located at least partially or entirely within an injector, syringe, or the like. The injector actuator sensor circuits 310 can thus detect a syringe actuation, and one or more characteristics of the syringe actuation, including but not limited to velocity and acceleration of actuation of the plunger of the syringe. the injector actuator sensor circuits 310 can include one or more electrical sensors, electromechanical sensors, microelectromechanical sensors (MEMS), or the like. As one example, a MEMS sensor within a syringe can be operatively coupled by the injector couplings 312 to the injector actuator sensors 310, where the injector actuator sensor circuits 310 can process the feedback provided by the MEMS sensor and generate a sensor feedback signal compatible with the injector interfaces 230 for transmission to the system processor 210.

The injector couplings 312 can operatively couple and physically couple at least one injector kit with the system 300. An injector kit can include syringe and one or more sensors operatively couplable with the injector couplings 312. The injector kit can include at least one syringe having a plunger and a breakable membrane, and can also include one or more sensors operatively coupled with, housed within, or integrated with, for example, one or more of the syringe, the plunger, and the breakable membrane. Thus, the injector kit can include structures corresponding to a medical syringe, and can include one or more sensor devices associated with one or more components of the medical syringe. The injector couplings 312 can include one or more connectors, interconnects, traces, switches, wires, ribbons, or cables, for example, to operatively couple the injector kit with the injector interfaces 230.

The injector structural sensor circuits 320 can detect feedback from at least one sensor operatively coupled with, integrated with, or the like, an injector device. The injector structural sensor circuits 320 can respond to modification, deformation, or breakage, for example, of at least portion of an injector. A portion of an injector can include, but is not limited to, a breakable membrane of a syringe as discussed above. The breakable membrane can include a barrier between substance in the syringe and an exterior of the syringe from the needle of the syringe. Thus, the breakable membrane can be disposed to at least partially block or slow, for example, flow of a substance from within the syringe into at least one of the needle of the syringe and a patient into whom the needle of the syringe is at least partially inserted or insertable. Deformation, displacement, or breakage, for example, of the plunger can be detected by a sensor among the injector structural sensor circuits 320 corresponding to the particular injector being operated by the medical provider. It is to be understood that the injector structural sensor circuits 320 can be located at least partially or entirely within the system 300, or can be located at least partially or entirely within an injector, syringe, or the like. It is to be understood that feedback is not limited to a medical provider. In addition to the examples discussed above, feedback can also be associated with, provided to, or the like, an automated system or a user. The feedback can includes temporal information on the stages of the injection relative to the cerebral sensor. Thus, the feedback can indicate the timing of the injection. The feedback can include volume of the injection or volume of injection administered or transferred to a patient.

The injector structural sensor circuits 320 can detect occurrence of an injection event originating from a particular syringe, and one or more characteristics of the injection event, including but not limited to force applied from the plunger to expel the substance from the syringe. As one example, the substance can include a mixture of air and saline. The injector structural sensor circuits 320 can include one or more electrical sensors, electromechanical sensors, microelectromechanical sensors (MEMS), or the like. As one example, a MEMS sensor within a syringe can be operatively coupled by the injector couplings 312 to the injector structural sensor circuits 320, where the injector structural sensor circuits 320 can process the feedback provided by the MEMS sensor and generate a sensor feedback signal compatible with the injector interfaces 230 for transmission to the system processor 210. The feedback can be based on a predetermined relationship between deformation or breakage of the breakable membrane of the syringe and force of injection can be stored at, detected by, calibrated by, or controlled by, for example, the injector structural sensor circuits 320, which can translate, or transform signal received to be compatible with the injector interfaces 230 as discussed above.

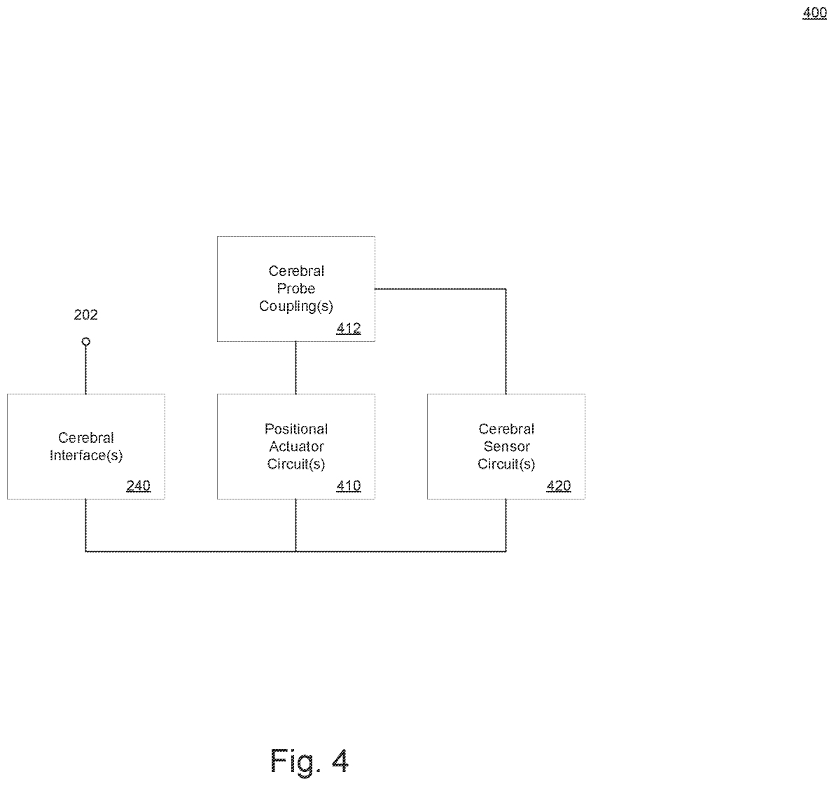

FIG. 4 illustrates a cerebral system further to the system architecture of FIG. 2. As illustrated by way of example in FIG. 4, a cerebral system 400 can include the cerebral interface 240, one or more positional actuator circuits 410, one or more cerebral probe couplings 412, and one or more cerebral sensor circuits 420.

The positional actuator circuits 410 can instruct one or more components of a sensor probe to modify at least one characteristic thereof. The characteristic can include a physical characteristic, including but not limited to absolute position within a particular coordinate space, a relative position with respect to an anatomical structure or reference position of the sensor probe or a particular component thereof, sensor sensitivity with respect to particular feedback characteristic of the TCD signal, sensor strength with respect to particular feedback characteristic of the TCD signal, and sensor beam shape. Thus, the positional actuator circuits 410 can advantageously provide instructions from at least the system processor 210 to control the sensor probe.

The cerebral probe couplings 412 can operatively couple and physically couple at least one sensor probe with the system 300. A sensor probe can correspond to the sensor probe 105. The cerebral probe couplings 412 can include one or more connectors, interconnects, traces, switches, wires, ribbons, or cables, for example, to operatively couple the sensor probe with the cerebral interfaces 230. The cerebral probe couplings 412 can include a particular communication channel, bus, or the like, for example, to effect bidirectional communication between at least the system processor 210 and the sensor probe 105.

The cerebral sensor circuits 420 can detect sensor feedback from the sensor probe, including by not limited to one or more components of a TCD signal. The cerebral sensor circuits 420 can include one or more communication channels, or the like, for example, to receive TCD signal components including M-mode, spectrogram, and envelope components from the cerebral probe couplings 412 and to transmit one or more of the components to the cerebral interface 240. The cerebral sensor circuits 420 can, in some implementations, receive a combined TCD signal and can transform the TCD signal or extract one or more components from the TCD signal to isolate or generate, for example, one or more of the M-mode, spectrogram, and envelope components. The cerebral sensor circuits 420 can receive raw unprocessed ultrasound data, including, but not limited to, IQ pairs.

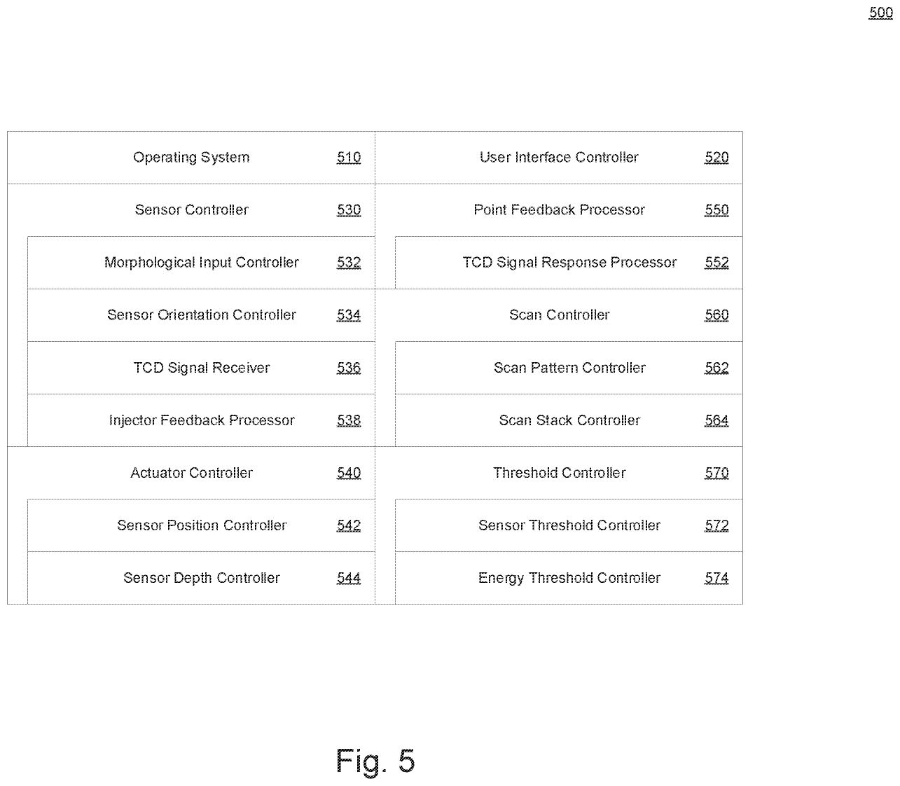

FIG. 5 illustrates a memory architecture, in accordance with present implementations. As illustrated by way of example in FIG. 5, a memory 500 can include an operating system 510, a user interface controller 520, a sensor controller 530, an actuator controller 540, a point feedback processor 550, a scan controller 560, and a threshold controller 570. The memory 500 can correspond to the system memory 220.

The operating system 510 can include hardware control instructions and program execution instructions. The operating system 510 can include a high level operating system, a server operating system, an embedded operating system, or a boot loader. The operating system 510 can include one or more instructions operable specifically with or only with the system processor 510. The user interface controller 520 can include hardware control instructions and program execution instructions to control or obtain feedback from one or more of the output device and 140 one or more of the input output device interfaces 250.

The sensor controller 530 can include one or more instructions to control a sensor, including but not limited to a sensor probe as discussed herein. The sensor controller 530 can control at least a physical position of a sensor probe and beam output of the sensor probe. The sensor controller 530 can include a morphological input controller 532, a sensor orientation controller 534, a TCD signal receiver 536, and an injector feedback processor 538.

The morphological input controller 532 can include one or more instructions to obtain one or more signal corresponding to a morphological structure. The morphological input controller 532 can generate at least one model corresponding to the morphological structure. Morphological structures can correspond to anatomical structures, and can include a cranial structure, portion thereof, or the like, for example, associated with a patient. The morphological input controller 532 can first identify an alternative sample volume and form a subset of the vascular map to reach the desired (diagnostically optimal) sample volume. The alternative sample volume can include an anatomical landmark including the Middle Cerebral Artery—Anterior Cerebral Artery (MCA-ACA) bifurcation.

The sensor orientation controller 534 can include one or more instructions to determine a position of a sensor probe or one or more components thereof. As one example, the sensor orientation controller 534 can include at least one positional sensor associated with at least one actuator corresponding to a particular sensor. As one example, the sensor orientation controller 534 can include at least one sensor associated with a displacement of at least one motor in an angular or linear coordinate space, for example.

The TCD signal receiver 536 can include one or more instructions to obtain at least one TCD signal from the sensor probe. The TCD signal receiver can also include at least one signal processing portion to extract a TCD signal from an electrical signal or analog signal, for example, received from the sensor probe.

The injector feedback processor 538 can include one or more instructions to obtain at least one signal from an injector kit or any component thereof. The injector feedback processor 538 can also include at least one signal processing portion to extract a TCD signal from an electrical signal or analog signal, for example, received from the sensor probe.

The actuator controller 540 can include one or more instructions to articulate at least one component of the sensor probe. The actuator controller 540 can include a sensor position controller 542, and a sensor depth controller 544.

The sensor position controller 542 can include one or more instructions to actuate a sensor probe with respect to a coordinate space or a morphological structure, for example. The sensor position controller 542 can provide instructions to actuate the sensor probe with respect to a coordinate system or a relative position of the sensor probe itself, including a default position of the sensor probe. The sensor position controller 542 can provide instructions to actuate the sensor probe with respect to a morphological structure, including a morphological model generated by the morphological input controller 532. The sensor position controller 542 can position the sensor probe with respect to a surface of a morphological structure, including, for example, skin of a patient's temple, head, or the like. The sensor depth controller 544 can include one or more instructions to actuate a sensor beam or the like of the sensor probe to detect feedback at a particular depth of a morphological structure below a surface of the morphological structure. As one example, the sensor depth controller 544 can instruct the sensor probe to emit a sensor beam at a depth corresponding to a particular vascular structure or the like.

The point feedback processor 550 can include one or more instructions to generate a point corresponding to a particular position and depth associated with the sensor probe. The point can be associated with a feedback value or values corresponding to a magnitude of feedback detected by the sensor probe at the position and depth. The point feedback processor 550 can include a TCD signal response processor 552. The TCD signal response processor 552 can include one or more instructions to obtain one or more TCD signals or signal components from the TCD signal. The TCD signal response processor 552 can identify and extract one or more of an M-mode component, a spectrogram component, and an envelope component from the TCD signal.

The scan controller 560 can include one or more instructions to store and retrieve one or more scan patterns associated with movement of a sensor probe, and to instruct the actuator controller to move the sensor probe in accordance with one or more scan patterns. For optimization purposes, the point feedback processor 550 can identify an optimized point based at least on an energy function that compares two signals, advantageously reducing the scope of optimization to finding the maximal value of the energy function. As one example, an energy function T can be computed on the measured TCD signal components of M-mode, spectrogram and envelope. A scan pattern can include a path of travel of a sensor probe with respect at least to a surface of a morphological structure. The path of travel can include one or more geometric shapes, and the scan controller 560 can include storage portion to store the scan patterns. As one example, a scan pattern can include at least one of a square, a circle, a triangle, a polygon, or the like. As one example, the scan pattern can include a spiral pattern in accordance with a shape. Thus, the scan pattern can include a single line around a center point. The scan controller can thus instruct the sensor probe to move in an expanding outward motion along a surface of a morphological structure, by for example, flowing a path in a circular spiral outward from a center point. The scan controller 560 can include a scan pattern controller 562 and a scan stack controller 564.

The scan pattern controller 562 can include one or more instructions to modify a scan pattern. The scan pattern controller 562 can modify a scan pattern in accordance with a resolution or scale, for example, associated with the scan being performed. T can be computed by visiting a location (x, y, z, Rx, Ry, d), sampling the signals, and computing T(x, y, z, Rx, Ry, d). The scan pattern controller 562 can command the interfaces of the system 300 to move the sensor probe in a pattern within a spatial region of interest and compute T, looking for the position that yields the largest measurement. The core iteration of this search scheme may vary, such as driven by a graduated search state-stack system. Searches can be pushed on or popped off the stack according to certain rules. The top search state can correspond to the currently active search and can also correspond to the highest-resolution search within the most localized area or volume, of all the searches in the stack. As one example, a scan can progress in higher degrees of granularity as a sensor probe finds increasingly optimal feedback at a particular position in the scan pattern path. As one example, the scan pattern controller 562 can instruct the sensor probe to conduct a scan along a first circular pattern having a first size. Once the threshold controller 570 indicates that an optimized point has been identified, the scan pattern controller 562 can replace the first circular pattern with a second circular pattern centered on the optimized point and having a second size. The second size can be smaller than the first size to enable the sensor probe to conduct a second scan along a scan pattern finer than the first scan pattern and to identify an optimized point with an increasing accuracy over an iterative scan process. As one example, the first size can be a diameter of a scan pattern corresponding to 10 mm, and a second size can be a diameter of a scan patter corresponding to 2 mm. It is to be understood that the sizes described herein are nonlimiting examples, and the scan pattern can be modified in size or shape in accordance with any iterative process to increase or decrease granularity.

The scan stack controller 564 can include one or more instructions to operate the scan controller to detect optimized points along multiple pathways and multiple scan patterns. The scan stack controller can include a stack object to store one or more optimized points. In response to detection of an optimized point with respect to a scan pattern, the scan stack controller 564 can push the optimized point to the stack object and can continue with the next iterative scan. If the scan controller 560 instructs the system to discard a particular iterative path through scanning at higher granularities, the scan controller 560 can pop the optimized points corresponding to that set of scans and continue searching from an optimized point lower in the stack object. The scan controller 560 can change the scan pattern associated with the scan in response, for example, to a pop operation to discard one or more optimized points.

For example, if a coarse spiral search, Sₙ, is being conducted, and a point of interest is discovered, then a fine spiral search Sₙ₊₁ is pushed onto the state stack. The fine spiral search can correspond to a resolution higher than the coarse spiral search, or can correspond to a size less than a size of the coarse spiral search. Size can correspond to area or volume of a search space. The fine spiral search can then be conducted centered at the point of interest provided by the coarse spiral search. If the fine spiral search finds another point of even higher value, then an even more refined search Sₙ₊₂ would be pushed onto the state stack and thus conducted. An energy threshold, as determined by an energy function computation, can increase as searches become more refined in terms of, for example, resolution or size. If, however, the fine spiral search Sₙ₊₁ does not uncover any points that meet its stricter signal threshold, then the fine spiral search can be “popped” off from the graduated search stack, and the coarse spiral search Sₙ can continue from where it left off, with its less strict threshold. Signals found by S′ can correspond to locally optimal solutions.

The threshold controller 570 can include one or more instructions to modify one or more thresholds associated with a scan and to evaluate one or more with respect to the thresholds. The threshold controller 570 can determine whether an optimized point satisfies a particular threshold, by comparing one or more values associated with the point to the threshold. The threshold values may be modulated using a gain or a penalty of either CBFV-derived values (e.g. morphological metrics such as slope, curvature, etc. or positional values such as X, Y, depth). The modulation of the threshold may be performed dynamically within an exam. The dynamic threshold may be additionally modulated using prior data collected during the exam. For example, the threshold can be based at least partially on comparing current CBFV or sensor probe position or orientation to one or more previously collected corresponding CBFV or sensor probe positions or orientations. Modulation with prior values can advantageously result in faster and more accurate discovery of an optimized point, to improve speed and accuracy or sensor probe calibration. More than one threshold or threshold mechanism may be used. As one example, a real-time threshold can be used to actively “find” a signal of interest and a second to assess the quality of the signal to be diagnostically adequate.

The threshold controller 570 can include a sensor threshold controller 572 and an energy threshold controller 574. The sensor threshold controller 572 can include one or more instructions to generate and enforce one or more thresholds associated with the sensor controller 530 or the actuator controller 540. The sensor threshold controller 572 can include thresholds to restrict movement or beam direction, or beam power, for example, of the sensor probe. Thus, the sensor threshold controller 572 can apply boundaries to the travel of the sensor probe in addition to the path set for the sensor probe's travel by the scan controller. The sensor threshold controller 572 can generate thresholds based on morphological structure of a patient's head to be scanned, or device constraints of the sensor probe or system, for example. The thresholds can be dynamic with respect to a scan resolution or size, a number of iterations of the scan controller 560, or a particular morphological structure, for example.

The energy threshold controller 574 can include one or more instructions to generate and modify thresholds corresponding to feedback associated with optimized points. The energy threshold controller 574 can designate a particular energy level associated with a TCD corresponding to, for example, a minimum magnitude of feedback associated with a particular point. The energy threshold controller 574 can include one or more energy thresholds, corresponding to one or more of an overall magnitude of response of a TCD signal at the point, or a magnitude of response of one or more components of the TCD signal. The components can include at least an M-mode component, a spectrogram component, and an envelope component. The energy threshold controller 574 can modify the energy threshold or thresholds in response to actions by the scan controller 560. As one example, the energy threshold controller 574 can increase an energy threshold in response to the scan pattern controller 562 modifying a scan pattern to a smaller size or higher resolution pattern.

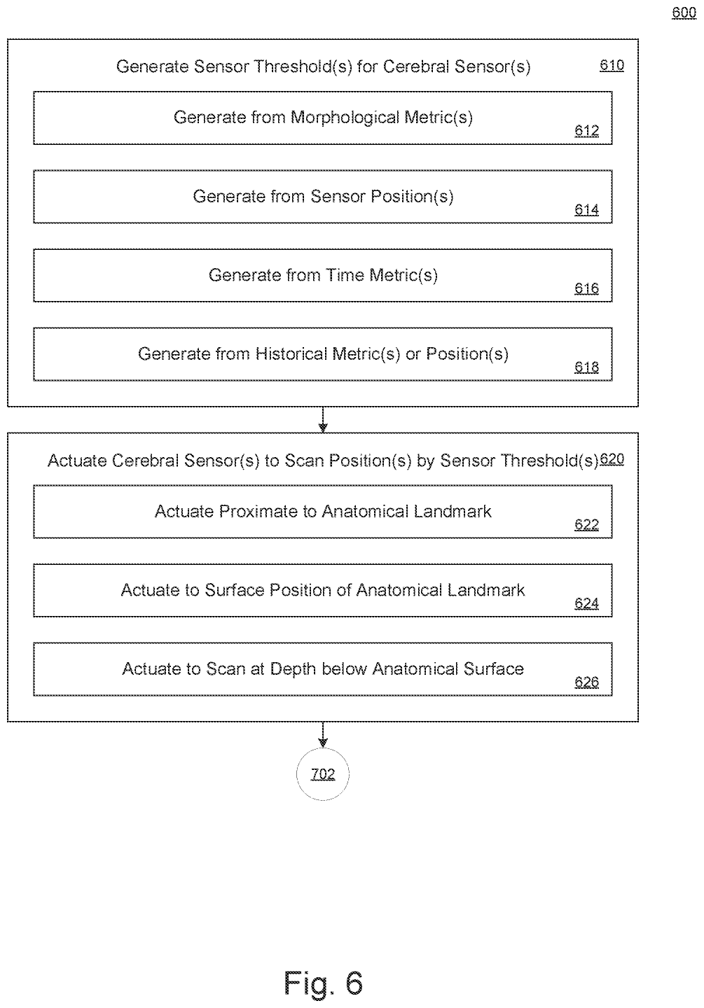

FIG. 6 illustrates a method of optimizing orientation of a cerebral probe, in accordance with present implementations. At least one of the systems 100-400 can perform method 600 according to present implementations. The method 600 can begin at 610.

At 610, the method can generate one or more sensor thresholds for one or more cerebral sensors. A sensor threshold can correspond to a constraint, guide, landmark, or boundary, for example, associated with at least one modifiable characteristics of a cranial sensor or component associated therewith. As one example, a sensor threshold can provide a maximum radial distance from a particular point along a surface of an anatomical structure or a depth into an anatomical structure from the surface thereof. The sensor threshold is not limited to a single threshold per sensor or group of sensors. As one example, the sensor threshold can include one or more distances from one or more landmarks, sensor positions, and the like. 610 can include at least one of 612, 614, 616 and 618. At 612, the method can generate the sensor threshold from one or more morphological metrics.

Morphological metrics can include values or representations, for example, associated with a feature of an anatomical structure. The features can include, for example one or more of a cranial ridge, cranial protrusion, surface cranial feature, surface organ feature, or the like. As one example, a surface organ feature can include an ear, nose, eye, or any portion thereof or associated therewith. At 614, the method can generate the sensor threshold from one or more sensor positions. The sensor positions can include any positions of any actuator or moveable component of the sensor, for example. A sensor position can include a position of a robotic arm in any lateral or angular orientation, and any configuration of a sensor beam, including but not limited to a beam width, beam distribution about a center axis of projection, and a beam intensity or power. At 616, the method can generate the sensor threshold from one or more time metrics. Time metrics can include one or more times, timestamp, or the like. The sensor threshold can vary based on time to, for example, grow or shrink an area or volume associated with a scan by a sensor probe with respect to time. The dynamic threshold described above may be additionally modulated by additional parameters, including but not limited to time elapsed or user preference. As one example, user preference can include pre-selecting a preference for quality of signal vs time of signal acquisition. The modification of a sensor threshold in accordance with a time metric can be associated with an exit condition. At 618, the method can generate the sensor threshold from one or more historical metrics or positions, or any combination thereof. The historical metrics or positions can include or be based on historical data for corresponding cranial senor operations. As one example, an initial sensor position can be based on historical pervious initial sensor positions resulting in successful identification of an optimized point for tracking by the cranial sensor. The historical metrics and positions can be transformed, modified, filtered, or the like, with respect to a similarity or difference between a current anatomical structure and historical anatomical structures associated with the historical metrics or positions. The historical anatomical structures can be corresponding anatomical structures of patients or subjects other than the current patient or subject undergoing a scan. The method 600 can then continue to 620.

At 620, the method can actuate the cerebral sensor to at least one scan position based at least partially on the sensor thresholds. 620 can include at least one of 622, 624 and 626. At 622, the method can actuate the cerebral sensor proximate to an anatomical landmark. At 624, the method can actuate the cerebral sensor to a surface position of an anatomical landmark. At 626, the method can actuate the cerebral sensor to a depth below an anatomical surface. The method 600 can then continue to 702.

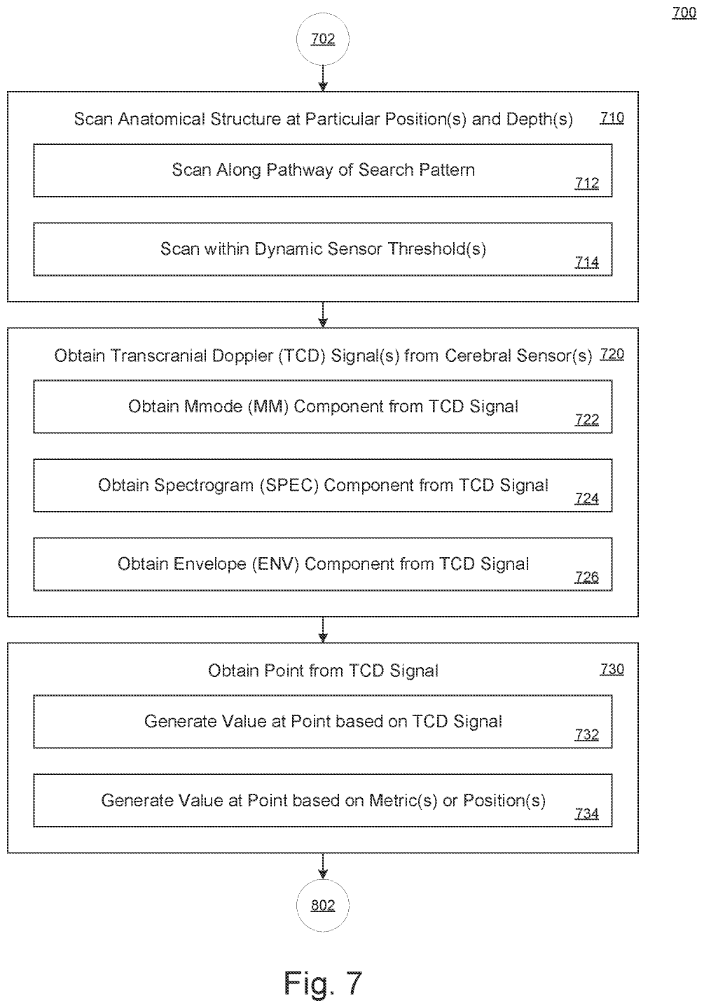

FIG. 7 illustrates a method of optimizing orientation of a cerebral probe, further to the method of FIG. 6. At least one of the systems 100-400 can perform method 700 according to present implementations. The method 700 can begin at 702. The method 700 can then continue to 710.

At 710, the method can scan an anatomical structure at a particular position and depth. A position can include, but is not limited to a linear position or an angular position. 710 can include at least one of 712 and 714. At 712, the method can scan along a pathway of a search pattern. The pathway can correspond to a path associated with the scan controller 560 or any component thereof. At 714, the method can scan within one or more dynamic sensor thresholds. Dynamic sensor thresholds can change based on one or more factors, and any combination of those factors. For example, the sensor thresholds can change based on time, as discussed above, feedback received from the sensor probe in the form of TCD signals or components, scan resolution associated with a particular iteration of a scan, or any combination thereof. The method 700 can then continue to 720.

At 720, the method can obtain at least one transcranial Doppler (“TCD”) signal from the cerebral sensor. The TCD signal can include one or more individually extractable and identifiable components. 720 can include at least one of 722, 724 and 726. At 722, the method can obtain at least one M-mode component from the TCD signal. At 724, the method can obtain at least one spectrogram component from the TCD signal. At 726, the method can obtain at least one envelope component from the TCD signal. The method 700 can then continue to 730.

At 730, the method can obtain at least one point from the TCD signal. The point can be a data structure, object, or the like, including at least one coordinate to spatially place the point with respect to an anatomical structure. The point can also be associated with a scan pattern, a scan iteration number or identifier, and a scan shape, for example. 730 can include at least one of 732 and 734. At 732, the method can generate at least one value at the obtained point, based on the TCD signal. At 734, the method can generate at least one value at the obtained point, based on at least one metric, or at least one position, or any combination thereof. The method 700 can then continue to 802.

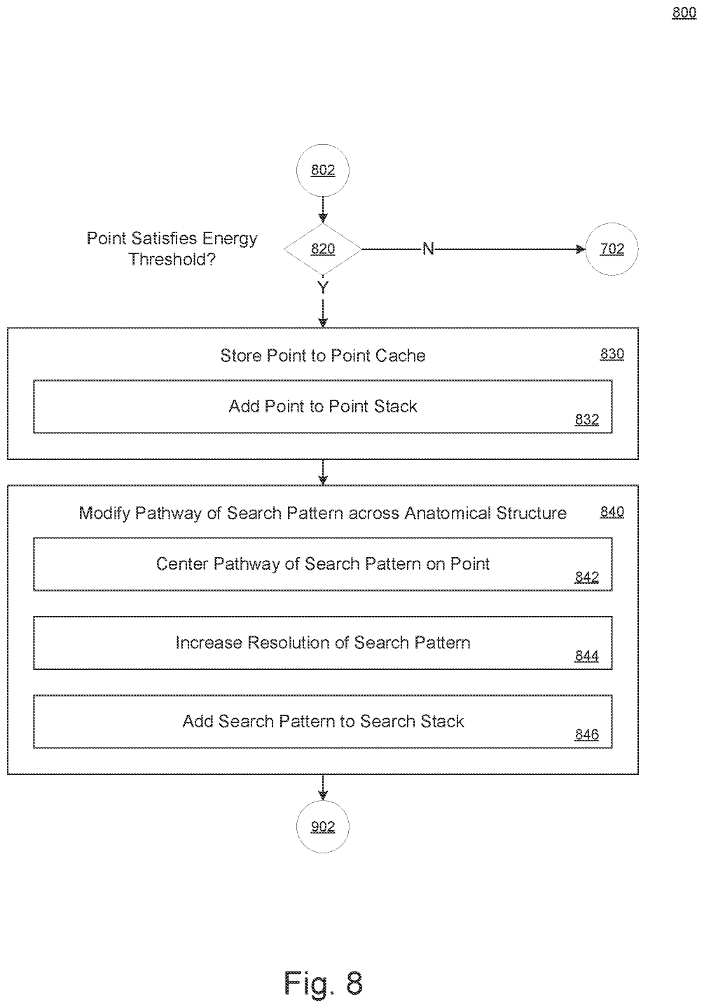

FIG. 8 illustrates a method of optimizing orientation of a cerebral probe, further to the method of FIG. 7. At least one of the systems 100-400 can perform method 800 according to present implementations. The method 800 can begin at 802. The method 800 can then continue to 810.

At 810, the method can determine whether an obtained point satisfies an energy threshold. An energy threshold can, correspond to energy associated with a TCD signal sensed at a sensor probe. The energy threshold can indicate a minimum energy threshold to indicate that a point can be considered an optimized point to obtain a TCD signal at the anatomical structure. The energy threshold can include a changing energy threshold that can, for example, increase to a higher threshold as resolution increases, to hone in iteratively on a strong TCD signal. In accordance with a determination that an obtained point satisfies an energy threshold, the method 800 can continue to 830. Alternatively, in accordance with a determination that an obtained point does not satisfy an energy threshold, the method 800 can continue to 702.

At 830, the method can store the obtained point to the point cache. 830 can 832. At 832, the method can add the point to a point stack. The point stack can correspond to the stack object of the scan stack controller 564. The method 800 can then continue to 840.

At 840, the method can modify a pathway of a search pattern across the anatomical structure. The pathway can be modified at least by the scan pattern controller 562 as discussed above. 840 can include at least one of 842, 844 and 846. At 842, the method can center the pathway of the search pattern on the obtained point. At 844, the method can increase a resolution of the search pattern. At 846, the method can add the search pattern to a search stack. The method 800 can then continue to 902.

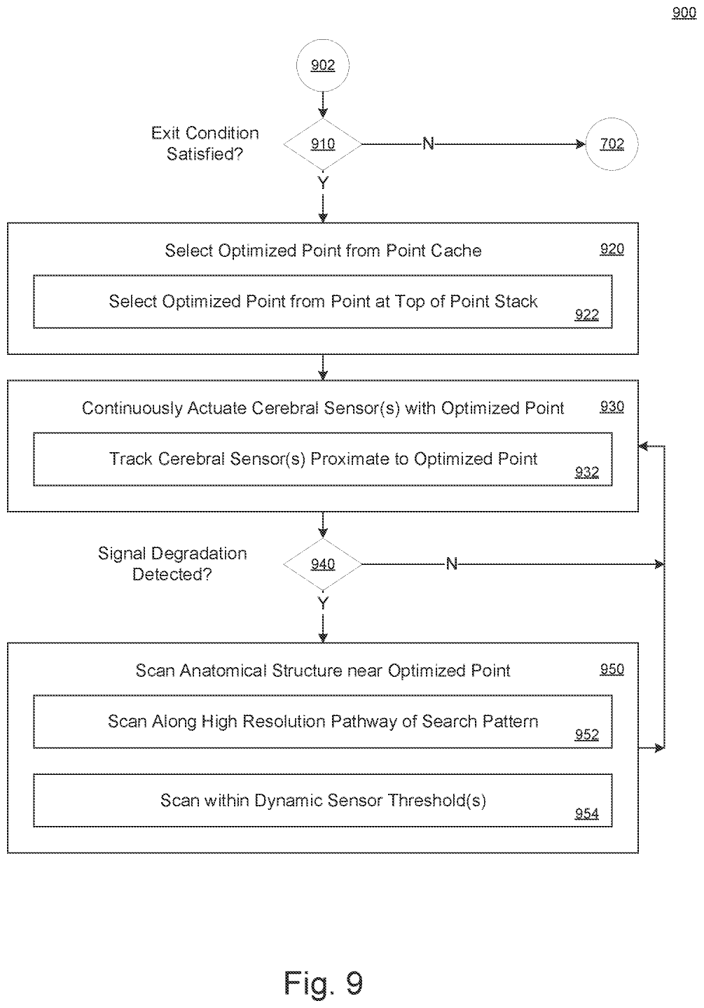

FIG. 9 illustrates a method of optimizing orientation of a cerebral probe, further to the method of FIG. 8. At least one of the systems 100-400 can perform method 900 according to present implementations. The method 900 can begin at 902. The method 900 can then continue to 910.

At 910, the method can determine whether an exit condition is satisfied. An exit condition can include a condition that a maximum number of iterations have passed, a maximum number of stack pops has been performed, a maximum number of stack pushes has been performed, and a particular TCD feedback strength has been detected, for example. In accordance with a determination that an exit condition is satisfied, the method 900 can continue to 920. Alternatively, in accordance with a determination that an exit condition is not satisfied, the method 900 can continue to 702.

At 920, the method can select an optimized point from the point cache. 920 can include 922. At 922, the method can select an optimized point from a point stored at a top of the point stack. The method 900 can then continue to 930.

At 930, the method can continuously actuate the cerebral sensor with the optimized point. The actuator controller 540 can operate cerebral sensor to remain stabilized proximate to the optimized point. The method 900 can then continue to 940.

At 940, the method can determine whether signal degradation is detected at the cerebral sensor. Signal degradation can occur in response to, for example, interference, or changes in biological activity at the anatomical structure, for example. Changes in blood pressure or circulation, for example, can modify the subsurface structure of the scan volume sufficiently to result in a degradation of signal. In accordance with a determination that signal degradation is detected at the cerebral sensor, the method 900 can continue to 950. Alternatively, in accordance with a determination that signal degradation is not detected at the cerebral sensor, the method 900 can continue to 930.

At 950, the method can scan the anatomical structure near the optimized point. The scan controller can perform a search along a search pattern at a higher level of granularity to recover an optimized point near the optimized point lost to signal degradation. The higher level of granularity can be, for example, the second size as discussed above. 950 can include at least one of 952 and 954. At 952, the method can scan along a high resolution pathway of the search pattern. At 954, the method can scan within one or more dynamic sensor thresholds. The method 900 can then continue to 930.

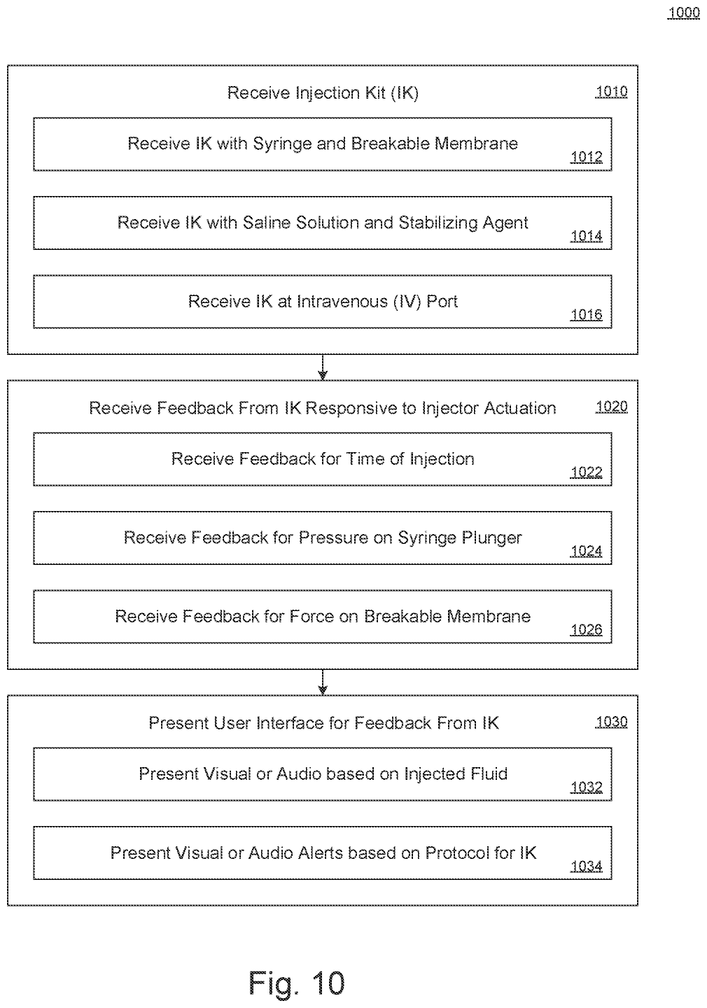

FIG. 10 illustrates a method of monitoring and generating feedback of an injection system, in accordance with present implementations. At least one of the systems 100-400 can perform method 1000 according to present implementations. The method 1000 can begin at 1010.

At 1010, the method can receive an injection kit. The injection kit can be a physical device connected by one or more wires, tubes, or the like, to a syringe and the system 300. The injection kit can be a removable or modular medical apparatus that can interface with the system 300. The injection kit can correspond to or include a single-shot kit. Single-shot kits can include a syringe with a saline solution and a stabilizing agent. The syringe can contain a pressure pocket, created by an in-line breakable membrane. The pressure pocket can maintain a positive or negative pressure of air compared to atmospheric pressure. A sudden pressure differential causing agitation of the fluid can occur when a user pushes or pulls on the syringe plunger, to release an injection of agitated microbubbles needed during an examination and sensor feedback procedure. The single shot injection system can include an integrated pressure or force sensor on the head of the plunger to measure or estimate the amount of force exerted by the user on the syringe, and a mechanical or optical encoder to estimate the dispensed volume to display the quality of the injection. The force sensor can indicate whether a vein was correctly punctured and if sufficient quantity of the agitated saline was injected.

1010 can include at least one of 1012, 1014 and 1016. At 1012, the method can receive an injection kit with a syringe and a breakable membrane. At 1014, the method can receive an injection kit with saline solution and a stabilizing agent. At 1016, the method can receive an injection kit at an intravenous port. The method 1000 can then continue to 1020.

At 1020, the method can receive feedback from the injection kit response to an actuation of the injector. 1020 can include at least one of 1022, 1024 and 1026. At 1022, the method can receive feedback corresponding to a time of injection. At 1024, the method can receive feedback corresponding to pressure on a plunger of a syringe of the injection kit. At 1026, the method can receive feedback corresponding to force on a breakable membrane of a syringe of the injection kit. The method 1000 can then continue to 1030.

At 1030, the method can present a user interface corresponding to feedback from the injection kit. The user interface controller can generate a presentation including one or more warnings, alerts, or the like associated with the feedback discussed above. A medical practitioner can advantageously modify performance of an injection based on the feedback to maintain or recover optimal injection to a patient to maximize TCD signal response at the anatomical structure. 1030 can include at least one of 1032 and 1034. At 1032, the method can present a visual presentation, an audio presentation, or any combination thereof. The display(s) and or speaker(s) may be configured to provide visual cues or audible cues, in the form of voice prompts to the user or patient for executing custom protocols, or confirmation that steps in the protocol are completed. Presentations can include sequential screens for baseline (without bubble injection), injection at rest, and injection during Valsalva Strain. The display can be configured to output at least one or more of the following estimates of a shunt (or absent shunt) such as bubble or emboli count, identification of regions of high intensity (indicating presence of shower or curtains) or shunts that are clinically significant (for example closable vs non-closable shunt, high risk/low risk shunt, etc., grades of shunt).

Video presentations can include a display indicating a numerical magnitude of force or pressure. As one example, the display can be configured to output a strength of a Valsalva strain performed by the user using change in Mean velocity (for example between rest and strain segments), change in pulsatility index, or change in waveform morphology. Waveform morphology can include a ratio of a second peak and a first peak of the CBFV waveform.

Audio presentations can include an audio sound generated in response to detection of force or pressure, or force or pressure satisfying a particular audio alert threshold. At 1034, the method can present visual alerts, audio alerts, or any combination thereof, based on a protocol associated with the injection kit. The system can send and receive alerts, prompts and notifications to subscribers or subscriber groups (e.g. care group of referring physician, treating physician, nurse, PCP, etc.). Such prompts, alerts and or notifications can be based on either pre-determined thresholds, custom thresholds, or based on prompting algorithms that can provide prompts or push notifications to user, patient, physicians, or other alerting subscribers for notifications of exam completion, results of exam, prompts for reviewing exam, etc. As one example, notifications can include alerts to a medical provider applying an injection to modify a speed of injection. As another example, notifications can be transmitted to a medical professional assigned to a patient, such as the primary care provider or specialist for the patient. Notifications regarding administration results or real-time progress of the administration of an injection can be transmitted to the assigned medical professional in response to injection activity from administration of the injection to the patient by a medical provider.

The alerting may be based on thresholds or values (e.g. shunt grade >=3), based on completed or ongoing events (e.g. prompting patient/user to hold breath after an injection) either visually or audibly, or using machine learning/natural-language processing methods for analyzing the comments and annotation in exam reports. The user interface controller 520 can present and or edit images, snapshots, videos, audios of the results and/or next treatment steps for facilitation of prompt and efficient patient treatment and/or care. The method 1000 can end at 1030.

Accurate diagnosis and treatment recommendation for the patient's vascular shunt condition can use at least one or more of the following methods. Automated grading of a right-to-left shunt can be quantified by how far a challenge section of an exam deviates from a baseline section of the exam by computing a direct difference between the two sections using raw or filtered versions of the M-mode and/or Spectrogram values.

Automated grading of a right-to left shunt by computing the moving difference between a baseline and challenge section is computed for the M-mode and/or spectrogram. Non-stationarity of the Doppler-shifted signal can be estimated as a measure of the number of bubbles in a challenge section. This can be done, for example, using wavelet transforms or other methods of measuring unexpected values in a time-varying system. Shunt grade can be estimated by training a model to recognize representative examples of shunting using at least one of the M-mode or Spectrogram. One example model can be constructed from a convolutional neural network with rectified linear units. Shunt grade can be estimated by training a model to recognize examples of the temporal changes that occur in a shunt. One example model can be constructed from a recurrent neural network. A sparse low range (SLR) decomposition method can be used to discriminate regions of high intensity in a spectrogram and/or M-mode from background, to compute showers in higher grade shunts. The shunt grade estimated can be correlated to recommendations for best treatment courses (such as surgical closure, prescription of anti-coagulants, etc.) based on classifications from prior outcome data and data presented to enable effective treatment/intervention decision for the patient.

The herein described subject matter sometimes illustrates different components contained within, or connected with, different other components. It is to be understood that such depicted architectures are illustrative, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mating and/or physically interacting components and/or wirelessly interactable and/or wirelessly interacting components and/or logically interacting and/or logically interactable components.

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

It will be further understood by those within the art that if a specific number of an introduced claim recitation is intended, such an intent will be explicitly recited in the claim, and in the absence of such recitation, no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general, such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Further, unless otherwise noted, the use of the words “approximate,” “about,” “around,” “substantially,” etc., mean plus or minus ten percent.

The foregoing description of illustrative implementations has been presented for purposes of illustration and of description. It is not intended to be exhaustive or limiting with respect to the precise form disclosed, and modifications and variations are possible in light of the above teachings or may be acquired from practice of the disclosed implementations. It is intended that the scope of the invention be defined by the claims appended hereto and their equivalents.

What is claimed is:

1. A method of calibrating orientation of a cerebral sensor and generating feedback from an injector for a cerebral sensor, the method comprising:
scanning an anatomical structure, by a cerebral sensor, proximate to the scan position and satisfying a first threshold;
obtaining at least one cranial signal from the cerebral sensor in accordance with a first pathway over the anatomical structure;
obtaining from the cranial signal a point corresponding to the anatomical structure;
storing the obtained point to a point cache operable to store one or more points, in response to a determination that the point satisfies a second threshold;
modifying the first pathway for actuating the cerebral sensor, in response to the determination that the obtained point satisfies the second threshold;
selecting an optimized point from the point stack; and
actuating the cerebral sensor based on the optimized point.

2. The method of claim 1, wherein the modifying the first pathway comprises:
generating a second pathway over the anatomical structure iteratively at a second granularity different from a first granularity associated with the first pathway.

3. The method of claim 1, wherein the first threshold corresponds to one or more metrics associated with one or more of a surface position and a depth of the scan position with respect to the anatomical structure.

4. The method of claim 1, wherein the first threshold corresponds to one or more metrics associated with the anatomical structure.

5. The method of claim 1, wherein the cranial signal comprises a transcranial Doppler signal.

6. The method of claim 1, wherein the second threshold corresponds to at least one of an M-mode component of the transcranial Doppler signal, a spectrogram component of the transcranial Doppler signal, an envelope component of the transcranial Doppler signal, and an IQ-pair component of the transcranial Doppler Signal.

7. The method of claim 1, further comprising:
receiving feedback from an injection kit, in response to an actuation of an injector associated with the anatomical structure; and
presenting, at a user interface, a presentation based on the feedback from the injection kit.

8. The method of claim 7, wherein the feedback comprises a pressure value corresponding to a magnitude of pressure applied to the injector.

9. The method of claim 7, wherein the presentation comprises an indication corresponding to a pressure applied to the injector.

10. A system of calibrating orientation of a cerebral sensor and generating feedback from an injector for a cerebral sensor, the system comprising:
a sensor controller to scan an anatomical structure, by a cerebral sensor, proximate to the scan position and satisfying a first threshold, obtain at least one cranial signal from the cerebral sensor in accordance with a first pathway over the anatomical structure, and actuate the cerebral sensor continuously based on an optimized point;
a point feedback processor operatively coupled with the sensor controller, the point feedback processor to obtain from the cranial signal a point corresponding to the anatomical structure, and to store the obtained point to a point cache operable to store one or more points, in response to a determination that the point satisfies a second threshold; and
a scan controller to modify the first pathway for actuating the cerebral sensor, in response to the determination that the obtained point satisfies the second threshold, and select the optimized point from the point stack.

11. The system of claim 10, wherein the scan controller further configured to:
generate a second pathway over the anatomical structure iteratively at a second granularity different from a first granularity associated with the first pathway.

12. The system of claim 10, wherein the first threshold corresponds to one or more metrics associated with one or more of a position and a depth of the scan position.

13. The system of claim 10, wherein the first threshold corresponds to one or more metrics associated with the anatomical structure.

14. The system of claim 10, wherein the cranial signal comprises a transcranial Doppler signal.

15. The system of claim 10, wherein the second threshold corresponds to at least one of an M-mode component of the transcranial Doppler signal, a spectrogram component of the transcranial Doppler signal, an envelope component of the transcranial Doppler signal, and an IQ-pair component of the transcranial Doppler Signal.

16. The system of claim 10, further comprising:
a user interface controller to present, at a user interface, a presentation based on feedback from an injection kit, and
the sensor controller being further configured to receive the feedback from the injection kit, in response to an actuation of an injector associated with the anatomical structure.

17. The system of claim 16, wherein the feedback comprises a pressure value corresponding to a magnitude of pressure applied to the injector.

18. The system of claim 16, wherein the presentation comprises an indication corresponding to a pressure applied to the injector.

19. A computer readable medium including one or more instructions stored thereon and executable by a processor to:
cause, by the processor, a cerebral sensor to scan an anatomical structure proximate to the scan position and satisfying a first threshold;
obtain, by the processor, at least one cranial signal from the cerebral sensor in accordance with a first pathway over the anatomical structure;
obtain, by the processor, from the cranial signal a point corresponding to the anatomical structure;
store, by the processor, the obtained point to a point cache operable to store one or more points, in response to a determination that the point satisfies a second threshold;
modify, by the processor, the first pathway for actuating the cerebral sensor, in response to the determination that the obtained point satisfies the second threshold;
select, by the processor, an optimized point from the point stack; and
actuate, by the processor, the cerebral sensor continuously based on the optimized point.

20. The computer readable medium of claim 19, wherein:
the first threshold corresponds to one or more metrics associated with one or more of a surface position of the scan position with respect to the anatomical structure, a depth of the scan position with respect to the anatomical structure;
the cranial signal comprises a transcranial Doppler signal; and
the second threshold corresponds to at least one of an M-mode component of the transcranial Doppler signal, a spectrogram component of the transcranial Doppler signal, an envelope component of the transcranial Doppler signal, and an IQ-pair component of the transcranial Doppler Signal.
